(19) United States
(12) Patent Application Publication
(10) Pub. No.: US 2018/0344168 A1
(43) Pub. Date: Dec. 6, 2018

(54) PHOTOACOUSTIC APPARATUS

(71) Applicant: CANON KABUSHIKI KAISHA, Tokyo (JP)

(72) Inventor: Naoto Abe, Machida-shi (JP)

(21) Appl. No.: 15/986,575

(22) Filed: May 22, 2018

(30) Foreign Application Priority Data
May 30, 2017 (JP) 2017-107062

(51) Int. Cl. A61B 5/00; A61B 5/145

(52) U.S. Cl. CPC A61B 5/0095; A61B 5/444; A61B 5/443; A61B 5/14546

(57) ABSTRACT

A plurality of semiconductor light-emitting elements radiates light onto a subject. An ultrasonic wave reception unit receives ultrasonic waves generated by the radiation of light onto the subject and outputs an electrical signal based on the received ultrasonic waves. A control unit controls light emission patterns of the plurality of semiconductor light-emitting elements to radiate the light onto the subject in respective different positions and at respective different times. An image generation unit generates a plurality of images, each such image being generated by independently reconstructing a respective electrical signal output by the ultrasonic wave reception unit based on the light radiated onto the subject in a respective one of the positions and at a respective one of the different times. The image generation unit generates a composite image concerning the subject by combining the plurality of images.

### BACKGROUND OF THE INVENTION

### Field of the Invention

Aspects of the present invention generally relate to a photoacoustic apparatus using a plurality of semiconductor light-emitting elements.

### Description of the Related Art

In recent years, as an imaging technology using light, photoacoustic apparatuses which perform imaging of the inside of a subject using the photoacoustic effect have been researched and developed. The photoacoustic apparatus forms an absorption coefficient distribution image based on ultrasonic waves (photoacoustic waves) that are generated by the photoacoustic effect from an optical absorber which has absorbed energy of light radiated onto a subject. Then, the photoacoustic apparatus generates a structure image or function image of the inside of the subject from the absorption coefficient distribution image.

To acquire information about a subject having a region broader than an irradiation spot of light, there is not only a method of performing scanning while changing the position of a light radiation unit but also a method of providing a plurality of light radiation units and sequentially switching a light radiation unit to radiate light.

Japanese Patent Application Laid-Open No. 2005-218684 discusses a configuration which guides light emitted from a light source to radiation units arranged in an array-like manner via a plurality of optical fibers and sequentially radiates light from the radiation units onto a subject. Moreover, the configuration discussed in Japanese Patent Application Laid-Open No. 2005-218684 generates an image of the subject based on photoacoustic waves that are generated by the subject being sequentially irradiated with light.

The configuration discussed in Japanese Patent Application Laid-Open No. 2005-218684 has a need for a number of optical fibers corresponding to the number of light radiation units, and is, therefore, hard to be available for high-density packaging. Moreover, it shows no findings for obtaining a good-quality image based on photoacoustic waves acquired by sequentially radiating light.

### SUMMARY OF THE INVENTION

According to an aspect of the present invention, a photoacoustic apparatus includes a plurality of semiconductor light-emitting elements, an ultrasonic wave reception unit, a control unit, and an image generation unit. The semiconductor light-emitting elements radiate light onto a subject. The ultrasonic wave reception unit receives ultrasonic waves generated by the radiation of light onto the subject and outputs an electrical signal based on the received ultrasonic waves. The control unit controls light emission patterns of the plurality of semiconductor light-emitting elements to radiate the light onto the subject in positions different from each other and at times different from each other. The image generation unit generates a plurality of images, each such image being generated by independently reconstructing a respective electrical signal output by the ultrasonic wave reception unit based on the light radiated onto the subject in a respective one of the positions and at a respective one of the times. The image generation unit generates a composite image concerning the subject by combining the plurality of images.

Further features of the present invention will become apparent from the following description of exemplary embodiments with reference to the attached drawings.

### BRIEF DESCRIPTION OF THE DRAWINGS

FIGS. 1A and 1B are schematic diagrams used to describe a photoacoustic apparatus according to a first exemplary embodiment of the present invention.

FIG. 2 is a block diagram of the photoacoustic apparatus according to the first exemplary embodiment.

FIGS. 3A, 3B, and 3C are diagrams illustrating a probe according to the first exemplary embodiment.

FIGS. 4A, 4B, 4C, and 4D are diagrams illustrating a light emission sequence of a plurality of semiconductor light-emitting elements and irradiated regions formed therewith in the first exemplary embodiment.

FIG. 5 is a diagram illustrating a specific configuration example of a computer in the first exemplary embodiment.

FIG. 6 is a timing chart in the first exemplary embodiment.

FIG. 7 is a timing chart in a second exemplary embodiment of the present invention.

FIGS. 8A and 8B are diagrams illustrating a light emission sequence of a plurality of semiconductor light-emitting elements and irradiated regions formed therewith in a second radiation mode in a third exemplary embodiment of the present invention.

FIG. 9 is a timing chart in the third exemplary embodiment.

FIGS. 10A, 10B, and 10C are diagrams illustrating a probe according to a fourth exemplary embodiment of the present invention.

FIGS. 11A, 11B, 11C, and 11D are diagrams illustrating a light emission sequence of a plurality of semiconductor light-emitting elements and irradiated regions formed therewith in the fourth exemplary embodiment.

### DESCRIPTION OF THE EMBODIMENTS

Hereinafter, various exemplary embodiments, features, and aspects of the invention will be described in detail below with reference to the drawings. In this regard, however, for example, the dimension, material, shape, and relative disposition of each constituent component described below can be changed or altered as appropriate according to the configuration of an apparatus to which the invention is applied and various conditions thereof. Therefore, the scope of the invention is not intended to be limited to the following description.

A photoacoustic apparatus according to an exemplary embodiment of the invention is related to a technique for generating and acquiring characteristic information about the inside of a subject by detecting acoustic waves propagating from the subject. Therefore, the invention is comprehended as a photoacoustic apparatus or a control method therefor, or a subject information acquisition method or signal processing method. The invention is also comprehended as a display method for generating and displaying an image representing characteristic information about the inside of a subject. The invention is also comprehended as a program that causes an information processing apparatus including hardware resources such as a central processing unit (CPU) and a memory to perform the above methods or a non-transitory computer-readable storage medium storing such a program.

The photoacoustic apparatus according to an exemplary embodiment of the invention includes a photoacoustic imaging apparatus using the photoacoustic effect, which receives photoacoustic waves generated inside a subject by light (electromagnetic waves) being radiated onto the subject and acquires characteristic information about the subject as image data. In this case, the characteristic information is information about characteristic values respectively corresponding to a plurality of positions inside the subject, generated with use of signals derived from the received photoacoustic waves.

In the present exemplary embodiment, photoacoustic image data is a concept including every piece of image data derived from photoacoustic waves generated by light irradiation. For example, the photoacoustic image data is image data representing a spatial distribution of at least one piece of subject information, such as the generated sound pressure (initial sound pressure), absorbed energy density, and absorption coefficient of photoacoustic waves, and the density (for example, oxygen saturation) of a material constituting a subject. Furthermore, photoacoustic image data representing spectral information, such as the density of a material constituting a subject, is obtained based on photoacoustic waves generated by light irradiation with a plurality of wavelengths different from each other. The photoacoustic image data representing spectral information can be an oxygen saturation, a value obtained by weighting the oxygen saturation with the density such as an absorption coefficient, a total hemoglobin concentration, an oxyhemoglobin concentration, or a deoxyhemoglobin concentration. Moreover, the photoacoustic image data representing spectral information can be a glucose concentration, a collagen concentration, a melanin concentration, or a volume fraction of fat or water.

A two-dimensional or three-dimensional characteristic information distribution is obtained based on pieces of characteristic information at various positions inside the subject. Distribution data can be generated as image data. The characteristic information can be obtained as not numerical data but distribution information at various positions inside the subject. In other words, the characteristic information can be distribution information, such as initial sound pressure distribution, energy absorption density distribution, absorption coefficient distribution, and oxygen saturation distribution.

In the present exemplary embodiment, the acoustic waves are typically ultrasonic waves, and include elastic waves called sound waves or acoustic waves. An electrical signal obtained by transducing acoustic waves with, for example, a transducer is also referred to as an “acoustic signal”. In this regard, however, the term “ultrasonic waves” or “acoustic waves” in the context of the present specification is not intended to limit wavelengths of such elastic waves. Acoustic waves generated by the photoacoustic effect are referred to as “photoacoustic waves” or “photoultrasonic waves”. An electrical signal derived from photoacoustic waves is also referred to as a “photoacoustic signal”. The distribution data is also referred to as “photoacoustic image data” or “reconstructed image data”.

In the following exemplary embodiments, a photoacoustic apparatus which irradiates a subject with pulsed light, receives photoacoustic waves from the subject, and generates a blood vessel image (structure image) of the inside of the subject is taken as an example of a subject information acquisition apparatus. While, in the following exemplary embodiments, a photoacoustic apparatus including a hand-held type probe is taken as an example, the following exemplary embodiments can also be applied to a photoacoustic apparatus which includes a probe mounted on a stage and performs mechanical scanning.

A photoacoustic apparatus according to an exemplary embodiment of the invention includes a plurality of semiconductor light-emitting elements, and an ultrasonic wave reception unit configured to receive ultrasonic waves generated by radiation of light from the plurality of semiconductor light-emitting elements onto a subject to output an electrical signal. The photoacoustic apparatus further includes an image generation unit configured to reconstruct an image concerning the subject based on the output electrical signal, and a control unit configured to control light emission patterns of the plurality of semiconductor light-emitting elements in such a manner that light is radiated from the plurality of semiconductor light-emitting elements onto the subject in positions different from each other and at times different from each other.

Then, the image generation unit generates a plurality of images by independently reconstructing a plurality of electrical signals each corresponding to the electrical signal derived from light radiated onto the subject in positions different from each other and at times different from each other, and generates a composite image by combining the plurality of images.

The photoacoustic apparatus according to the present exemplary embodiment is configured to include a light radiation unit containing a plurality of semiconductor light-emitting elements, and, therefore, facilitates achievement of high-density packaging. Moreover, since the photoacoustic apparatus according to the present exemplary embodiment generates a plurality of images by independently reconstructing a plurality of electrical signals derived from light radiated onto the subject in positions different from each other and at times different from each other, noises derived from light radiated in the other positions and at the other times are unlikely to enter each image. Therefore, a good-quality image can be obtained.

Furthermore, it is desirable that the image generation unit perform weighting processing during generation of a composite image based on information concerning a light quantity distribution of an irradiated region of light on the subject, which is determined, at least, based on the light emission pattern. In a plurality of light emission patterns, a region in which irradiated regions of light overlap becomes larger in light quantity than a region in which irradiated regions of light do not overlap. Therefore, the image generation unit performs correction processing for performing weighting corresponding to the light quantity distribution, so that an influence of a difference in light quantity distribution on an image can be reduced.

Moreover, the image generation unit can determine a region in which to reconstruct an image, based on information concerning a light quantity distribution of an irradiated region of light on the subject corresponding to the light emission pattern.

Moreover, it is desirable that the control unit be configured to enable light emission in radiation modes differing from each other in sequence of light emission patterns. To enable light emission in radiation modes different from each other, the control unit is able to cause a plurality of semiconductor light-emitting elements to perform light emission in a light emission pattern corresponding to each radiation mode. Furthermore, at least one of the radiation modes can be set as a light emission pattern in which all of the plurality of semiconductor light-emitting elements performs light emission. At that time, radiation modes can be switched by a mode control unit. During switching of the radiation modes, a radiation mode (light emission patterns) can be determined based on information concerning an instruction from the user or an optical absorption coefficient of the surface of the subject (for example, the skin of a living body). For example, in a case where the optical absorption coefficient of a surface of the subject is smaller than a predetermined value, the mode control unit is able to select a radiation mode in which all of the plurality of semiconductor light-emitting elements performs light emission. The predetermined value can be set based on, for example, the color of a skin.

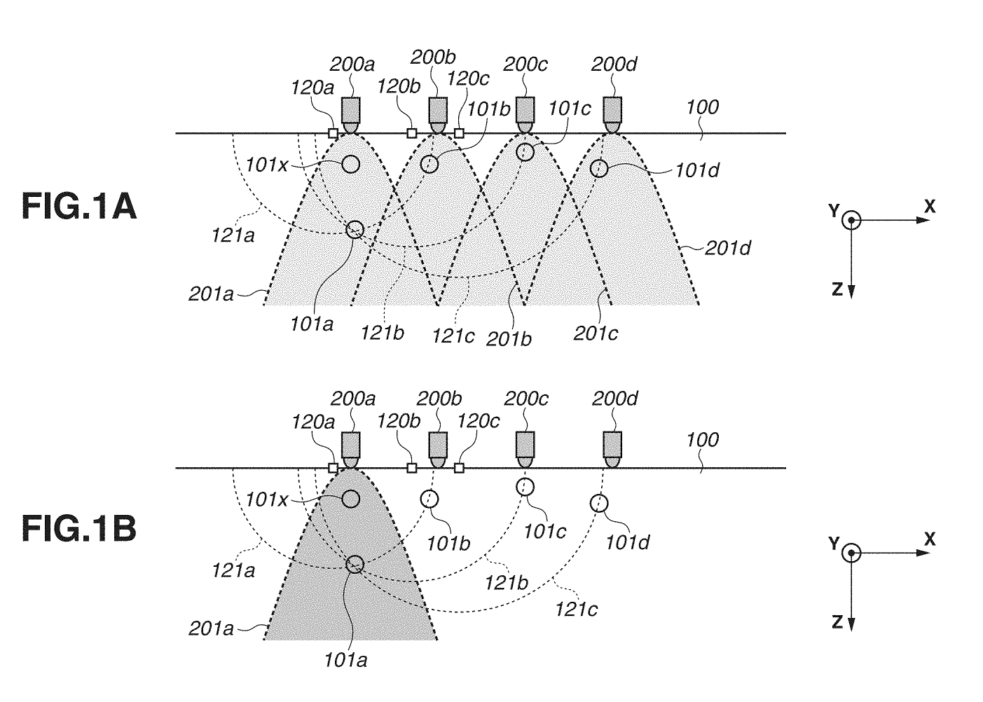

FIGS. 1A and 1B are schematic diagrams used to describe a photoacoustic apparatus according to a first exemplary embodiment of the present invention.

FIG. 1A illustrates a subject 100 and optical absorbers 101a, 101b, 101c, 101d, and 101x contained in the subject 100. The position of each optical absorber is merely an example. The optical absorber is, for example, oxyhemoglobin, deoxyhemoglobin, a blood vessel containing a lot of such hemoglobin, a new blood vessel formed near the tumor, or melanin of the pigment contained in the skin. Then, the optical absorber receives radiation of light and generates photoacoustic waves (ultrasonic waves). Transducers 120a, 120b, and 120c, which are included in an ultrasonic wave reception unit 120, convert photoacoustic waves into respective electrical signals. The ultrasonic wave reception unit 120 has the transducers 120a, 120b, and 120c arranged side by side in an array-like manner. A part of each of the transducers 120a, 120b, and 120c is illustrated in a schematic manner for ease of description. XZ cross-sections 121a, 121b, and 121c are isochronous surfaces in which the transducers 120a, 120b, and 120c receive photoacoustic waves generated at the optical absorber 101a at the same time. The photoacoustic waves generated on the isochronous surfaces 121a, 121b, and 121c cannot be separately converted into electrical signals when being received by the transducers 120a, 120b, and 120c. Semiconductor light-emitting elements 200a, 200b, 200c, and 200d constitute a light radiation unit 200. The semiconductor light-emitting elements 200a, 200b, 200c, and 200d radiate light with irradiated regions 201a, 201b, 201c, and 201d, respectively, which are schematically illustrated in FIG. 1A. The irradiated region of light is scattered inside the subject and is generally distributed in a more diffuse manner.

Referring to FIG. 1A, photoacoustic waves generated by the optical absorber 101a are focused on and described in more detail. The photoacoustic waves generated by the optical absorber 101a are received and converted into an electrical signal by the transducer 120a. However, when receiving photoacoustic waves generated by the optical absorber 101a, the transducer 120a also concurrently receives photoacoustic waves generated by the optical absorber 101b, which is located on the isochronous surface 121a. The optical absorber 101b generating photoacoustic waves is close to the semiconductor light-emitting element 200b and is thus located at a place in which the energy of radiated light is large, as apparent from the irradiated region 201a. Therefore, the photoacoustic waves generated by the optical absorber 101b becomes larger than the photoacoustic waves generated by the optical absorber 101a. Accordingly, in a case where an electrical signal obtained from the optical absorber 101a is small, the electrical signal may be masked by an electrical signal obtained from the optical absorber 101b and thus may become unrecognizable. Moreover, in a case where the state of radiation by the semiconductor light-emitting element 200b onto the subject is changed due to a change in contact state between the hand-held type probe and the subject, the amplitude of large photoacoustic waves generated by the optical absorber 101b changes and becomes noises which vary relative to the photoacoustic waves generated by the optical absorber 101a. In this way, the photoacoustic waves generated by the optical absorber 101b at a region in which the energy of radiation by the semiconductor light-emitting element 200b, which is located on the isochronous surface 121a of the transducer 120a, is large become a cause for noise with respect to the photoacoustic waves generated by the optical absorber 101a.

Moreover, in a case where the optical absorber 101b is located in front of or behind the isochronous surface 121a of the transducer 120a, the large photoacoustic waves generated by the optical absorber 101b are received before or after the photoacoustic waves generated by the optical absorber 101a. As a result, after reconstruction, a signal which is actually non-existent is generated in front of or behind the optical absorber 101a, and thus also becomes a large cause for noise.

On the other hand, photoacoustic waves generated by the optical absorber 101x, which is located at a place in which the energy of radiation by the semiconductor light-emitting element 200a is large, are not present on the isochronous surface 121a of the transducer 120a with respect to the photoacoustic waves generated by the optical absorber 101a. Therefore, a photoacoustic signal obtained by conversion performed by the transducer 120a is able to be easily separated based on a difference in reception time.

Next, a case where photoacoustic waves generated by the optical absorber 101a are received and converted into an electrical signal by the transducer 120b or 120c is described. Photoacoustic waves generated by the optical absorber 101c at a region in which the energy of radiation by the semiconductor light-emitting element 200c, which is located on the isochronous surface 121b of the transducer 120b, is large become a cause for noise with respect to the photoacoustic waves generated by the optical absorber 101a. Moreover, photoacoustic waves generated by the optical absorber 101d at a region in which the energy of radiation by the semiconductor light-emitting element 200d, which is located on the isochronous surface 121c of the transducer 120c, is large become a cause for noise with respect to the photoacoustic waves generated by the optical absorber 101a.

As described above, with respect to photoacoustic waves generated by an optical absorber of interest, which are received by a transducer of the ultrasonic wave reception unit 120, photoacoustic waves having a large intensity generated by an optical absorber located on the isochronous surface and close to a semiconductor light-emitting element become a cause for noise.

As mentioned above, photoacoustic waves generated by an optical absorber located near the skin surface close to a semiconductor light-emitting element have a great influence. For example, in a case where the amount of melanin of the pigment contained in the skin is large, photoacoustic waves generated at the skin surface are large, so that the above-mentioned cause for noise becomes large. Moreover, for example, a mole and body hair also become a cause for noise.

From this, it can be understood that, to reduce a cause for noise, it would be good that, as illustrated in FIG. 1B, only the semiconductor light-emitting element 200a performs light emission with regard to an irradiated region formed by radiation performed by the semiconductor light-emitting element 200a and the ultrasonic wave reception unit 120 receives photoacoustic waves. More specifically, with respect to photoacoustic waves generated by the optical absorber 101a, the optical absorber 101b, which is located on the isochronous surface 121a of the transducer 120a, seldom or never generates photoacoustic waves because the semiconductor light-emitting element 200b does not perform light emission. As a result, photoacoustic waves generated by the optical absorber 101b seldom or never affect photoacoustic waves generated by the optical absorber 101a. Similarly, with respect to photoacoustic waves generated by the optical absorber 101a, the optical absorber 101c, which is located on the isochronous surface 121b of the transducer 120b, seldom or never generates photoacoustic waves because the semiconductor light-emitting element 200c does not perform light emission. As a result, photoacoustic waves generated by the optical absorber 101c seldom or never affect photoacoustic waves generated by the optical absorber 101a. Moreover, with respect to photoacoustic waves generated by the optical absorber 101a, the optical absorber 101d, which is located on the isochronous surface 121c of the transducer 120c, seldom or never generates photoacoustic waves because the semiconductor light-emitting element 200d does not perform light emission. As a result, photoacoustic waves generated by the optical absorber 101d seldom or never affect photoacoustic waves generated by the optical absorber 101a.

In this way, turning off semiconductor light-emitting elements other than a semiconductor light-emitting element corresponding to a region (irradiated region) in which to reconstruct an image (generate a reconstructed image) enables reducing influences of optical absorbers located near the other semiconductor light-emitting elements. Naturally, in this case, photoacoustic waves are seldom or never generated in the irradiated regions corresponding to the semiconductor light-emitting elements turned off, so that it is naturally hard to acquire images formed with the photoacoustic waves. Accordingly, only the semiconductor light-emitting element 200a is caused to perform light emission, so that a reconstructed image is generated with respect to the irradiated region 201a corresponding to the semiconductor light-emitting element 200a. Subsequently, only the semiconductor light-emitting element 200b is caused to perform light emission, so that a reconstructed image is generated with respect to the irradiated region 201b corresponding to the semiconductor light-emitting element 200b, and, subsequently, only the semiconductor light-emitting element 200c is caused to perform light emission, so that a reconstructed image is generated with respect to the irradiated region 201c corresponding to the semiconductor light-emitting element 200c. Subsequently, only the semiconductor light-emitting element 200d is caused to perform light emission, so that a reconstructed image is generated with respect to the irradiated region 201d corresponding to the semiconductor light-emitting element 200d. Finally, the respective acquired reconstructed images are combined to acquire a reconstructed image of the entire subject.

This enables acquiring a reconstructed image having a good image quality over the entire region of the subject. On the other hand, since radiation is performed four times to obtain a reconstructed image of the entire subject, approximately a quadruple time is required to obtain the reconstructed image.

### <Apparatus Configuration>

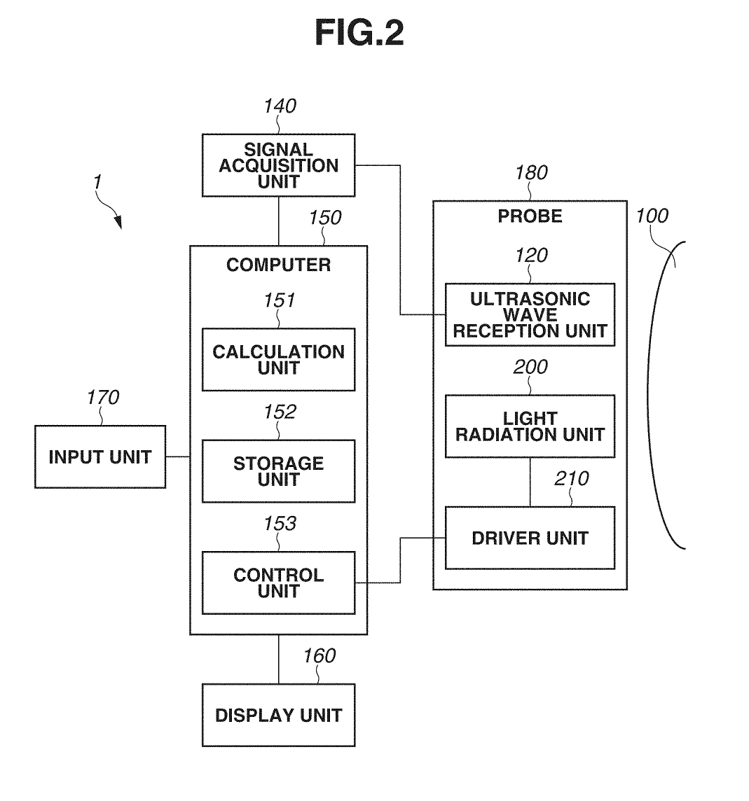

FIG. 2 is a block diagram of the photoacoustic apparatus 1 according to the present exemplary embodiment, which implements the above-described operation. Hereinafter, a configuration of the photoacoustic apparatus 1 according to the present exemplary embodiment is described with reference to the block diagram of FIG. 2. The photoacoustic apparatus 1 includes a probe 180, a signal acquisition unit 140, a computer 150, a display unit 160, and an input unit 170. The probe 180 includes a light radiation unit 200, a driver unit 210, and an ultrasonic wave reception unit 120. The computer 150 includes a calculation unit 151, a storage unit 152, and a control unit 153.

The driver unit 210 controls light emission of a plurality of semiconductor light-emitting elements of the light radiation unit 200 according to a radiation pattern (light emission pattern) of a radiation mode, which is described below. Details of the method for controlling light emission of a plurality of semiconductor light-emitting elements of the light radiation unit 200 are described below.

The semiconductor light-emitting elements of the light radiation unit 200 perform light emission with a first period (sampling period) according to a radiation pattern, thus irradiating a subject 100. The ultrasonic wave reception unit 120 receives photoacoustic waves generated from the subject 100 with the first period (sampling period), thus outputting an electrical signal (photoacoustic signal) as an analog signal. The signal acquisition unit 140 converts the analog signal output from the ultrasonic wave reception unit 120 into a digital signal, thus outputting the digital signal to the computer 150. The computer 150 calculates, with a second period (the period of image capturing frame rate), an arithmetic mean of the digital signal output from the signal acquisition unit 140 with the first period (sampling period), and stores the arithmetic mean as an electrical signal derived from photoacoustic waves (photoacoustic signal) in a memory. The computer 150 generates photoacoustic image data by performing processing, such as image reconstruction, on the stored digital signal. Then, the photoacoustic image data is displayed by the display unit 160.

Moreover, the computer 150 performs control of the entire photoacoustic apparatus 1.

Although not illustrated, the computer 150 can perform image processing for displaying or processing for synthesizing a graphic for graphical user interface (GUI) on the obtained photoacoustic image data.

While the present exemplary embodiment is described with use of the terms “first period (sampling period)” and “second period (the period of image capturing frame rate), the “period” used in exemplary embodiments of the invention does not need to be “perfectly constant in repetition time”. In other words, in the exemplary embodiments of the invention, even in a case where repetition is performed at time intervals that are not constant, the term “period” is used. Moreover, the first period (sampling period) includes, for example, a period in which a break period is included. A repetition time in a time that does not include the break period is referred to as a “period” in the exemplary embodiments of the invention.

The user (for example, a doctor or technician) can perform a diagnosis by checking a photoacoustic image displayed on the display unit 160. The displayed image can be stored in, for example, a memory included in the computer 150 or a data management system connected to the photoacoustic apparatus 1 via a network, based on a storing instruction from the user or the computer 150. The input unit 170 receives, for example, an instruction from the user.

### <Detailed Configuration of Each Block>

Subsequently, a desirable configuration of each block is described in detail.

### <Probe 180>

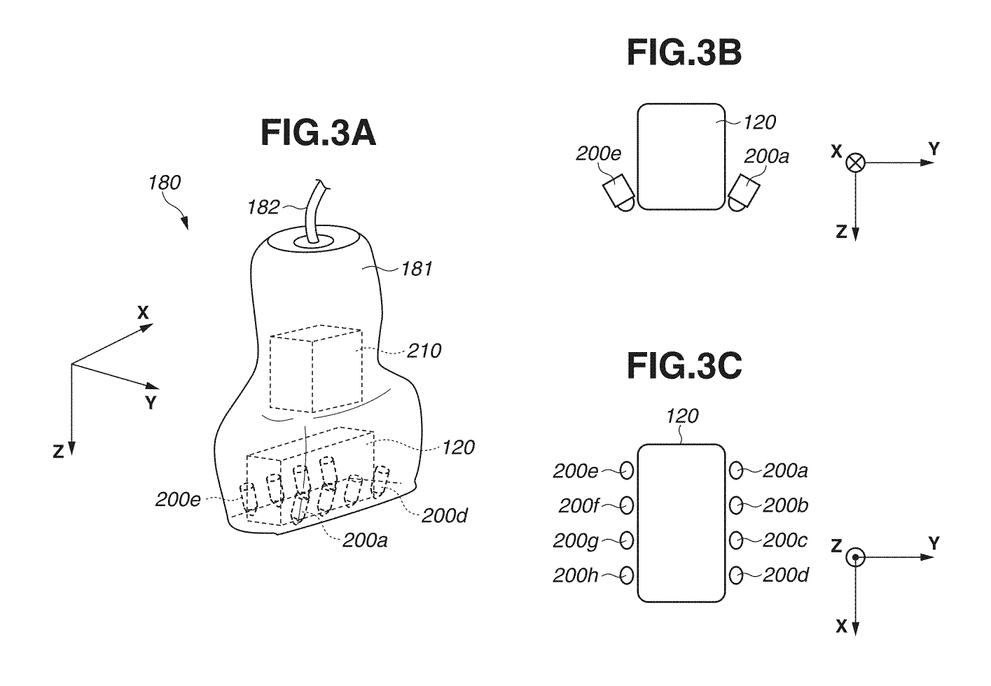

FIGS. 3A, 3B, and 3C are diagrams illustrating a configuration of the photoacoustic apparatus 180, which is a hand-held type probe according to the first exemplary embodiment of the invention. In the following description, the photoacoustic apparatus 180 is simply referred to as a “probe 180”.

Referring to FIG. 3A, the probe 180 includes a light radiation unit 200, a driver unit 210, an ultrasonic wave reception unit 120, and a housing 181. The housing 181 is a casing which encloses the light radiation unit 200, the driver unit 210, and the ultrasonic wave reception unit 120. The user can grip the housing 181 to use the probe 180 as a hand-held type probe. The light radiation unit 200 radiates light pulses onto a subject. The light radiation unit 200 is configured with, for example, a plurality of semiconductor light-emitting elements. As illustrated in FIGS. 3A to 3C, in the first exemplary embodiment of the invention, eight semiconductor light-emitting elements (for example, semiconductor lasers) 200a to 200h are used to configure the light radiation unit 200. The configuration of the light radiation unit 200 is not limited to this configuration, but can be, for example, a configuration including four semiconductor light-emitting elements (semiconductor lasers) 200a to 200d or can be a configuration including thirty-two semiconductor light-emitting elements (semiconductor lasers). The type or number of semiconductor light-emitting elements is determined based on a specified quantity of light. Furthermore, the X-, Y-, and Z-axes in FIGS. 3A to 3C indicate coordinate axes in a case where the probe is left to stand, and are not the axes which limit the orientation of the probe during use thereof.

The probe 180, which is illustrated in FIG. 3A, is connected to the signal acquisition unit 140 via a cable 182. The cable 182 includes a wiring used to supply electric power to the light radiation unit 200, a light emission control signal wiring, and a wiring used to output an analog signal output from the ultrasonic wave reception unit 120 to the signal acquisition unit 140. The cable 182 can be provided with a connector in such a way as to have a configuration in which the probe 180 can be separated from the other constituent components of the photoacoustic apparatus.

FIG. 3B is a Y-Z sectional view of a portion including the light radiation unit 200 and the ultrasonic wave reception unit 120 of the probe 180. FIG. 3C is a diagram of the portion including the light radiation unit 200 and the ultrasonic wave reception unit 120 of the probe 180 as viewed from a contact surface with the subject. Referring to FIG. 3C, semiconductor light-emitting elements are arranged in an array-like manner along the X-direction (first direction). Furthermore, the ultrasonic wave reception unit 120 can be configured to include a plurality of ultrasonic transducers, which can be arranged in an array-like manner along the X-direction (first direction).

### <Light Radiation Unit 200>

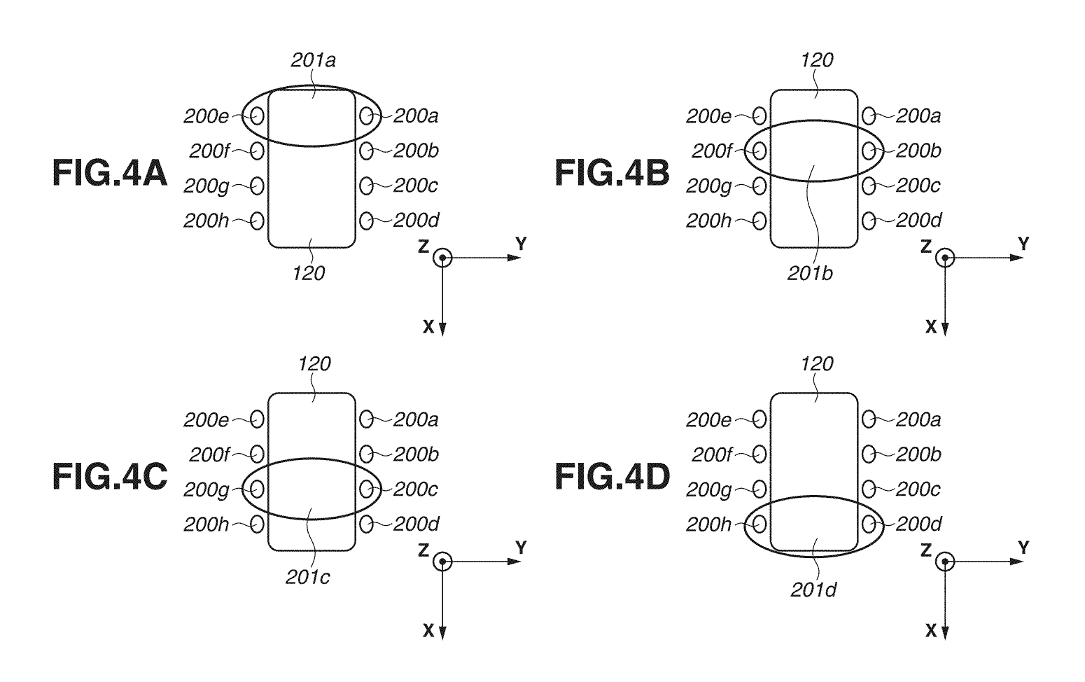

As illustrated in FIGS. 3A to 3C, the light radiation unit 200 is configured with eight semiconductor light-emitting elements 200a to 200h. Eight semiconductor light-emitting elements 200a to 200h are mounted at both sides of the ultrasonic wave reception unit 120, which is configured with a transducer array. Each of pairs of the semiconductor light-emitting elements 200a and 200e, the semiconductor light-emitting elements 200b and 200f, the semiconductor light-emitting elements 200c and 200g, and the semiconductor light-emitting elements 200d and 200h, which are mounted at both sides of the ultrasonic wave reception unit 120, performs light emission at the same time, thus radiating light into the subject at a portion just below the ultrasonic wave reception unit 120. A light emission sequence of the semiconductor light-emitting elements 200a to 200h and irradiated regions thereof are illustrated in FIGS. 4A, 4B, 4C, and 4D.

The light radiation unit 200 generates light to be radiated onto the subject 100. To generate pulsed light and acquire a material density such as oxygen saturation, the light radiation unit 200 is desirably a light source capable of outputting a plurality of wavelengths.

Moreover, from the quantity of light specified as a light source or usage of mounting inside the housing of the probe 180, it is desirable that the light radiation unit 200 include a plurality of semiconductor light-emitting elements, such as semiconductor lasers or light-emitting diodes, as illustrated in FIGS. 3A to 3C. Outputting a plurality of wavelengths can be implemented by performing switching light emission using a plurality of types of semiconductor lasers or light-emitting diodes which generate light with different wavelengths.

The pulse width of light which the light radiation unit 200 emits is, for example, 10 nanoseconds (ns) or more and 1 microsecond (μs) or less. Moreover, the wavelength of light is desirably 400 nanometers (nm) or more and 1600 nm or less, but the wavelength can be determined according to optical absorption characteristics of an optical absorber intended to be imaged. To perform imaging of a blood vessel at high resolution, wavelengths large in absorption at a blood vessel (400 nm or more and 800 nm or less) can be used. To perform imaging of the deep portion of a living body, light having wavelengths small in absorption at background tissues (for example, water and fat) of the living body (700 nm or more and 1100 nm or less) can be used. In the present exemplary embodiment, since semiconductor light-emitting elements are used as a light source of the light radiation unit 200, the quantity of light is insufficient. In other words, a photoacoustic signal which is obtained by performing radiation once does not reach an intended signal-to-noise ratio (S/N). Therefore, with respect to each order of the light emission sequence, light emission is performed with a first period (sampling period), an arithmetic mean of the photoacoustic signal is calculated to improve S/N, and, then, a reconstructed image is calculated with a second period (the period of image capturing frame rate) based on the calculated arithmetic mean of the photoacoustic signal.

In the case of a use application for which outputs of the semiconductor light-emitting elements are sufficient, light emission with a first period (sampling period) and calculation of an arithmetic mean of the photoacoustic signal do not need to be performed. In other words, a reconstructed image can be generated with radiation performed once.

An example of the wavelength of the light radiation unit 200 used in the present exemplary embodiment is desirably a wavelength of 797 nm. In other words, the exemplary wavelength is a wavelength capable of reaching the deep portion of a subject, and is suitable for detection of a blood vessel structure because absorption coefficients of oxyhemoglobin and deoxyhemoglobin are approximately equal. Moreover, if a light source with a wavelength of 756 nm is used as the second wavelength, an oxygen saturation can be obtained by using an absorption coefficient difference between oxyhemoglobin and deoxyhemoglobin.

### <Ultrasonic Wave Reception Unit 120>

The ultrasonic wave reception unit 120 includes ultrasonic transducers, each of which receives photoacoustic waves generated by light emission performed with the first period (sampling period) to output an electrical signal, and a supporting member, which supports the ultrasonic transducers. In the following description, the ultrasonic transducer is simply referred to as a “transducer”. As a member configuring the transducer, for example, a transducer using, for example, a piezoelectric material and a capacitance-type transducer Fabry-Perot interferometer can be used. The piezoelectric material includes, for example, a piezo-ceramic material, such as piezoelectric zirconate titanate (PZT), and a high-molecular piezoelectric membrane material, such as polyvinylidene fluoride (PVDF). The capacitance-type transducer is referred to as a “capacitive micro-machined ultrasonic transducer (CMUT)”.

An electrical signal obtained by a transducer with the first period (sampling period) is a time-resolved signal. Therefore, the amplitude of the electrical signal represents a value that is based on a sound pressure received by a transducer at each time (for example, a value proportional to the sound pressure).

Furthermore, the transducer is desirably the one capable of detecting frequency components configuring photoacoustic waves (typically, 100 kilohertz (kHz) to 10 megahertz (MHz)). Moreover, it is also desirable that a plurality of transducers be arranged side by side on the supporting member to form a flat surface or curved surface such as that called a 1D array, a 1.5D array, a 1.75D array, or a 2D array. Furthermore, in FIGS. 3A to 3C, a 1D array of transducers is schematically illustrated.

The ultrasonic wave reception unit 120 can include an amplifier which amplifies time-series analog signals output from the transducers. Moreover, the ultrasonic wave reception unit 120 can include an analog-to-digital (A/D) converter which converts time-series analog signals output from the transducers into time-series digital signals. In other words, the ultrasonic wave reception unit 120 can include the signal acquisition unit 140.

Furthermore, to detect acoustic waves from various angles to improve image resolution, such a transducer arrangement as to surround the subject 100 from all of the sides is desirable. Moreover, in a case where the subject 100 is too large to be surrounded from all of the sides, the transducers can be arranged on a hemispherical supporting member. A probe 180 including an ultrasonic wave reception unit 120 having such a shape is not a hand-held type probe, and is suitable for a mechanical-scanning type photoacoustic apparatus, which relatively moves the probe with respect to the subject 100. The movement of the probe can be performed with use of a scanning unit such as an XY stage. Furthermore, the arrangement and number of transducers and the shape of the supporting member are not limited to those mentioned above, and can be optimized according to the subject 100.

A medium which propagates photoacoustic waves can be arranged in a space between the ultrasonic wave reception unit 120 and the subject 100. This enables matching of acoustic impedances at a surface boundary between the subject 100 and the transducers. The medium includes, for example, water, oil, and ultrasonic gel.

The photoacoustic apparatus 1 can include a holding member which holds the subject 100 to stabilize the shape thereof. The holding member is desirably a member which is high in both light transmittivity and acoustic wave transmittivity. For example, polymethylpentene, polyethylene terephthalate, and acrylic can be used.

In a case where the apparatus according to the present exemplary embodiment not only generates a photoacoustic image but also generates an ultrasound image by transmission and reception of acoustic waves, the transducer can also function as a transmission unit that transmits acoustic waves. A transducer serving as a reception unit and a transducer serving as a transmission unit can be a single (common) transducer or can be separate configurations.

### <Signal Acquisition Unit 140>

The signal acquisition unit 140 includes amplifiers, each of which amplifies an electrical signal that is an analog signal output from the ultrasonic wave reception unit 120 and generated with the light emission performed with the first period (sampling period), and A/D converters, each of which converts the analog signal output from the amplifier into a digital signal. The signal acquisition unit 140 can be configured with, for example, a Field Programmable Gate Array (FPGA) chip.

An operation of the signal acquisition unit 140 is described in more detail. Analog signals output from a plurality of transducers arranged in an array-like manner of the ultrasonic wave reception unit 120 are amplified by the respective corresponding amplifiers and are then converted by the respective corresponding A/D converters into digital signals. The A/D conversion rate corresponds to at least two times the bandwidth of an input signal or more. As mentioned above, if the frequency components of photoacoustic waves are at 100 kHz to 10 MHz, the A/D conversion rate corresponds to a conversion performed at a frequency of 20 MHz or more, desirably, at a frequency of 40 MHz. Furthermore, the signal acquisition unit 140 uses a light emission control signal to synchronize timing of light radiation and timing of signal acquisition processing. In other words, A/D conversion is started at the above-mentioned A/D conversion rate based on the light emission time with every first period (sampling period), and the obtained analog signal is converted into a digital signal. As a result, a digital data string at every time interval of one out of the A/D conversion rate (at every A/D conversion interval) from the light emission time is able to be acquired with every first period (sampling period) from each of the plurality of transducers.

The signal acquisition unit 140 is also called a “data acquisition system (DAS)”. In the context of the present application, the electrical signal is a concept including not only an analog signal but also a digital signal.

As mentioned above, the signal acquisition unit 140 can be mounted inside the housing 181 of the probe 180. With such a configuration, information between the probe 180 and the computer 150 is transferred with a digital signal, so that noise resistance is improved. Moreover, as compared with the case of transferring an analog signal, using a high-speed digital signal enables reducing the number of wirings, so that the operability of the probe 180 is improved.

Moreover, an arithmetic mean operation to be described below can also be performed by the signal acquisition unit 140. In this case, it is desirable that the arithmetic mean operation be performed with use of hardware such as a FPGA.

### <Computer 150>

The computer 150 includes a calculation unit (image generation unit) 151, a storage unit 152, and a control unit 153. A unit assuming a calculation function as the calculation unit 151 can be configured with a processor, such as a CPU or a graphics processing unit (GPU), or an arithmetic circuit, such as an FPGA chip. These units can be configured with a single processor or arithmetic circuit, or can be configured with a plurality of processors or arithmetic circuits.

The computer 150 performs an arithmetic mean operation described below with respect to each of the plurality of transducers. The computer 150 performs an arithmetic mean operation on every piece of data of the same time from the light emission time of the above-mentioned digital data string output from the signal acquisition unit 140 with every first period (sampling period). Then, the computer 150 stores, in the storage unit 152, the arithmetic-mean digital data string as an arithmetic-mean electrical signal (photoacoustic signal) derived from photoacoustic waves with every second period (the period of image capturing frame rate).

Then, the calculation unit 151 performs generation of photoacoustic image data (a structure image or a function image) using image reconstruction based on the arithmetic-mean photoacoustic signal stored in the storage unit 152 with every second period (the period of image capturing frame rate), and performs other various calculation processing operations. The calculation unit 151 can receive, from the input unit 170, various parameter inputs, such as the speed of sound of the subject and a configuration of the holding portion, and use the parameter inputs for the calculation operations.

A reconstruction algorithm with which the calculation unit 151 converts the electrical signal into three-dimensional volume data can employ an optional method, such as a time-domain back projection method, a Fourier domain back projection method, and a model-based method (repetitive calculation method). The time-domain back projection method includes, for example, universal back-projection (UBP), filtered back-projection (FBP), and phasing and summing (delay-and-sum).

In a case where the light radiation unit 200 employs two wavelengths, the calculation unit 151 performs image reconstruction processing to generate a first initial sound pressure distribution from a photoacoustic signal derived from light of the first wavelength and to generate a second initial sound pressure distribution from a photoacoustic signal derived from light of the second wavelength. Moreover, the calculation unit 151 obtains a first absorption coefficient distribution by correcting the first initial sound pressure distribution with a light quantity distribution of the light of the first wavelength and obtains a second absorption coefficient distribution by correcting the second initial sound pressure distribution with a light quantity distribution of the light of the second wavelength. Additionally, the calculation unit 151 obtains an oxygen saturation distribution from the first and second absorption coefficient distributions. Furthermore, as long as the oxygen saturation distribution is eventually obtained, the contents or orders of calculation operations are not limited to those mentioned above.

The storage unit 152 is configured with a non-transitory storage medium, such as a volatile memory including a random access memory (RAM), and a read-only memory (ROM), a magnetic disc, and a flash memory. Furthermore, a storage medium storing a program is a non-transitory storage medium. Additionally, the storage unit 152 is configured with a plurality of storage media.

The storage unit 152 is able to store various pieces of data, such as photoacoustic signals subjected to the arithmetic mean operation with the second period (the period of image capturing frame rate), photoacoustic image data generated by the calculation unit 151, and reconstructed image data that is based on photoacoustic image data.

The control unit 153 is configured with an arithmetic element such as a CPU. The control unit 153 controls an operation of each constituent component of the photoacoustic apparatus 1. The control unit 153 stores a plurality of radiation patterns and radiation modes described below, and sends, to the driver unit 210, a light emission control signal used to control light emission of the semiconductor light-emitting elements with the first period (sampling period) according to a plurality of radiation patterns of the designated radiation mode. Then, the semiconductor light-emitting elements perform light emission according to a plurality of radiation patterns of the designated radiation mode, thus irradiating the subject. The control unit 153 also serves as a light emission control unit which controls light emission according to radiation patterns of a radiation mode. Moreover, the control unit 153 can have the function to select a radiation mode during acquisition of a reconstructed image from among a plurality of radiation modes according to an instruction from the user or automatically, as described below.

Moreover, the control unit 153 reads out program code stored in the storage unit 152, and controls an operation of each constituent component of the photoacoustic apparatus 1 based on the program code.

Additionally, the control unit 153 performs, for example, adjustment of an image with respect to the display unit 160. With this, oxygen saturation distribution images are sequentially displayed along with the movement and photoacoustic measurement of the probe 180.

The computer 150 can be a workstation exclusively designed for the present exemplary embodiment. The computer 150 can also be a general-purpose personal computer (PC) or workstation which are configured to operate according to instructions from a program stored in the storage unit 152. Moreover, the components of the computer 150 can be configured with respective different pieces of hardware. Additionally, at least some constituent components of the computer 150 can be configured with a single piece of hardware.

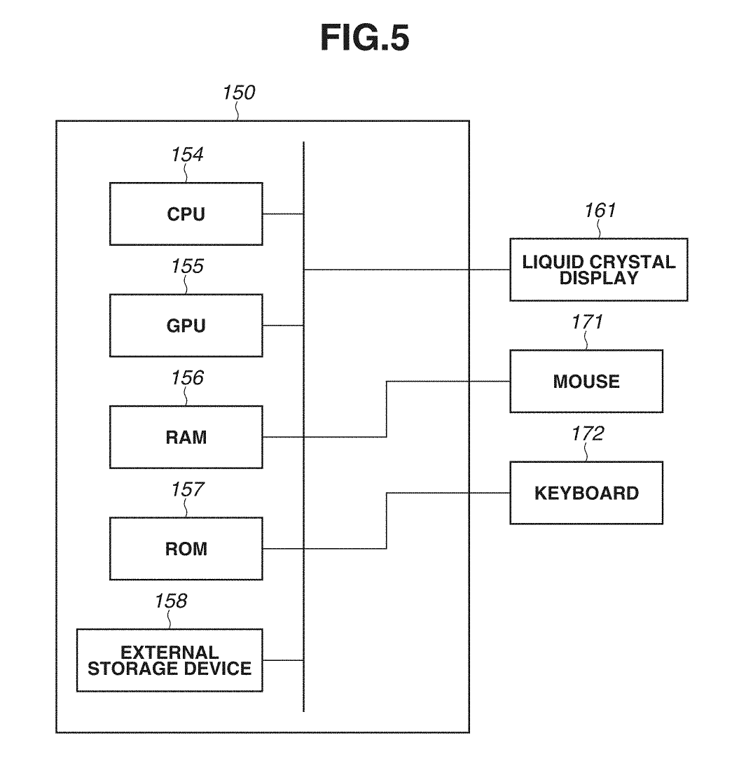

FIG. 5 illustrates a specific configuration example of the computer 150 according to the present exemplary embodiment. The computer 150 according to the present exemplary embodiment includes a CPU 154, a GPU 155, a RAM 156, a ROM 157, and an external storage device 158. Moreover, a liquid crystal display 161, which serves as the display unit 160, and a mouse 171 and a keyboard 172, which serve as the input unit 170, are connected to the computer 150.

Moreover, the computer 150 and the ultrasonic wave reception unit 120 can be provided as a configuration contained in a common casing. Additionally, some signal processing operations can be performed by a computer contained in a casing and the remaining signal processing operations can be performed by a computer provided outside the casing. In this case, the computers provided inside and outside the casing can be collectively referred to as a computer according to the present exemplary embodiment. In other words, pieces of hardware constituting a computer do not need to be contained in a single casing. An information processing apparatus provided in, for example, a cloud computing service and installed in a remote location can be used as the computer 150.

The computer 150 is equivalent to a processing unit in the present exemplary embodiment. In particular, the calculation unit 151 plays a central role in implementing the function of the processing unit.

### <Display Unit 160>

The display unit 160 is a display such as a liquid crystal display or an organic electroluminescence (EL) display. The display unit 160 is a device which displays, for example, an image that is based on, for example, subject information obtained by the computer 150 and numerical values of a specific position. The display unit 160 can also display a graphical user interface (GUI) used to operate an image or the apparatus. Image processing (for example, adjustment of a luminance value) can be performed by the display unit 160 or the computer 150.

### <Input Unit 170>

An operation console which is able to be operated by the user and is configured with, for example, a mouse and a keyboard can be employed as the input unit 170. Moreover, the display unit 160 can be configured with a touch panel, so that the display unit 160 can be used as the input unit 170. The input unit 170 receives inputs, such as instructions and numerical values, from the user, and transmits the inputs to the computer 150.

Furthermore, the constituent components of the photoacoustic apparatus can be configured as respective separate apparatuses or can be configured as a single integrated apparatus. Moreover, at least some constituent components of the photoacoustic apparatus can be configured as a single integrated apparatus.

Moreover, the computer 150 also causes the control unit 153 to perform drive control of constituent components included in the photoacoustic apparatus. Additionally, the display unit 160 can display, in addition to an image generated by the computer 150, for example, a GUI. The input unit 170 is configured to allow the user to input information thereto. The user can use the input unit 170 to perform operations for starting and ending of measurement, designation of a radiation mode described below, and an instruction for storage of a generated image.

### <Subject 100>

The subject 100 is not a component constituting the photoacoustic apparatus, but is described below. The photoacoustic apparatus according to the present exemplary embodiment is able to be used for the purpose of, for example, diagnosis of, for example, malignant tumor or blood vessel disease of a human being or an animal or follow-up of chemical treatment. Therefore, the subject 100 is assumed to be a region targeted for diagnosis, such as a living body, specifically, a breast, each organ, a network of vessels, a head, a neck, an abdomen, and extremities including hands and fingers and toes of a human body or an animal. For example, if a human body is an object to be measured, for example, oxyhemoglobin, deoxyhemoglobin, a blood vessel containing a lot of such hemoglobin, or a new blood vessel formed near a tumor can be set as a target serving as an optical absorber. Moreover, for example, plaque on the wall of a carotid artery can be set as a target serving as an optical absorber. If the subject is a human body, melanin of the pigment contained in the skin may become the above-mentioned optical absorber which generates photoacoustic waves that become a cause for noise. Moreover, a pigment such as methylene blue (MB) or indocyanine Green (ICG), gold fine particles, or an externally-introduced material obtained by integrating or chemically modifying those can be set as an optical absorber. Additionally, a puncture needle or an optical absorber applied to a puncture needle can be set as an observation object. The subject can be an inanimate object such as a phantom or a test object.

### <Operation of Exemplary Embodiment>

The operation of causing the semiconductor light-emitting elements to sequentially perform light emission to acquire a reconstructed image as described above is described as follows. In the example illustrated in FIGS. 4A to 4D, each of pairs of the semiconductor light-emitting elements 200a and 200e, the semiconductor light-emitting elements 200b and 200f, the semiconductor light-emitting elements 200c and 200g, and the semiconductor light-emitting elements 200d and 200h performs light emission at the same time, and these pairs perform light emission in turns. In exemplary embodiments of the invention, each of light emission states illustrated in FIGS. 4A to 4D is referred to as a “radiation pattern”, and a set of radiation patterns is referred to as a “radiation mode”. Thus, the radiation mode illustrated in FIGS. 4A to 4D is made up of a repetition of four radiation patterns. Naturally, the number of semiconductor light-emitting elements or the number of radiation patterns for use in exemplary embodiments of the invention is not limited to that number. Exemplary embodiments of the invention can be applied whatever number can be employed.

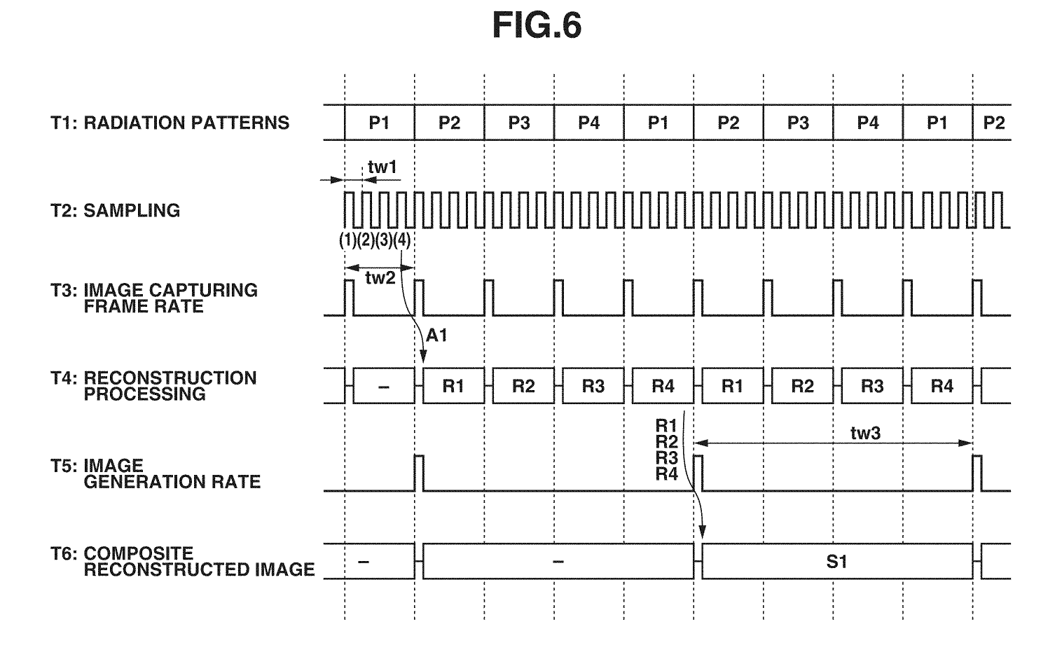

FIG. 6 is a timing chart used to comprehensibly describe an operation in the first exemplary embodiment of the invention. In FIG. 6, the horizontal axis is a time axis. Control of these timings is performed by the computer 150, an FPGA, or dedicated hardware. The method of acquiring a photoacoustic signal in the photoacoustic apparatus and generating a photoacoustic image that is based on the acquired photoacoustic signal according to the present exemplary embodiment is described in detail with reference to FIG. 6.

To implement a radiation mode illustrated in FIGS. 4A to 4D, the photoacoustic apparatus determines and switches radiation patterns in which a plurality of semiconductor light-emitting elements of the light radiation unit 200 performs light emission, as indicated on line T1 illustrated in FIG. 6. More specifically, the radiation pattern P1 is a pattern in which the semiconductor light-emitting elements 200a and 200e perform light emission, and the radiation pattern P2 is a pattern in which the semiconductor light-emitting elements 200b and 200f perform light emission. Moreover, the radiation pattern P3 is a pattern in which the semiconductor light-emitting elements 200c and 200g perform light emission, and the radiation pattern P4 is a pattern in which the semiconductor light-emitting elements 200d and 200h perform light emission. The photoacoustic apparatus has a radiation mode in which these four radiation patterns are repeated.

Since the quantity of light of each semiconductor light-emitting element is small, with a view to improving S/N, as indicated on line T2 illustrated in FIG. 6, the photoacoustic apparatus causes the light radiation unit 200 to perform light emission in the respective radiation patterns with a first period (sampling period tw1), and acquires a photoacoustic signal caused by light emission with the sampling period tw1.

Furthermore, the length of the sampling period tw1 is set in consideration of a maximum permissible exposure (MPE) with respect to the skin. This is because, the shorter the length of the sampling period tw1, the smaller the MPE value becomes. For example, in a case where the measurement wavelength is 750 nm, the pulse width of pulsed light is 1 μs, and the sampling period tw1 is 0.1 milliseconds (ms), the MPE value with respect to the skin is about 14 J/m². On the other hand, in a case where the peak power of pulsed light radiated from the light radiation unit 200 is 2 kilowatts (kW) and the irradiation area from the light radiation unit 200 is 150 mm², the light energy radiated from the light radiation unit 200 onto the subject 100, such as a human body, is about 13.3 J/m². In this case, the light energy radiated from the light radiation unit 200 becomes equal to or less than the MPE value. In this way, if the sampling period tw1 is 0.1 ms or more, it can be assured that the light energy is equal to or less than the MPE value. In the above-described way, the light energy is set in a range that does not exceed the MPE value, based on the sampling period tw1, the peak power of pulsed light, and the irradiation area.

Next, as indicated on line T2 to line T4 illustrated in FIG. 6, the photoacoustic apparatus acquires a photoacoustic signal four times with the sampling period tw1 (photoacoustic signals (1) to (4)) and then performs an arithmetic mean operation on the photoacoustic signals, thus acquiring an arithmetic-mean photoacoustic signal A1 with every period of image capturing frame rate tw2. The arithmetic mean to be used here includes, for example, simple average, moving average, and weighted average. For example, In a case where the time of the sampling period tw1 is 0.1 ms and the image generation rate is 60 hertz (Hz), the period of image generation rate tw3 is 16.7 ms and the period of image capturing frame rate tw2 is 4.17 ms. In this case, within the period of image capturing frame rate, the number of times of the arithmetic mean operation is able to be set to 41 times.

Next, as indicated on line T4 illustrated in FIG. 6, the photoacoustic apparatus performs the above-mentioned processing for reconstruction based on the arithmetic-mean photoacoustic signal A1, thus obtaining reconstructed image data R1. Reconstructed image data is sequentially calculated with the period of image capturing frame rate. To perform calculations for reconstruction processing, the photoacoustic apparatus can perform reconstruction processing of the entire subject region for every radiation in each radiation pattern. However, in a case where reconstruction processing is performed on a large region, the amount of calculation becomes large, so that a long time is required for calculations. To reduce the amount of calculation, the photoacoustic apparatus can determine a region on which to perform reconstruction processing according to an irradiated region in each radiation pattern. For example, when setting regions on which to perform reconstruction processing, the photoacoustic apparatus can determine a threshold value for the quantity of light of an irradiated region in each radiation pattern in such a manner that there appear regions in which regions on which to perform reconstruction processing according to radiation patterns overlap. Then, as indicated on line T4 to line T6 illustrated in FIG. 6, the photoacoustic apparatus combines reconstructed image data R1 to reconstructed image data R4, thus sequentially calculating a composite reconstructed image with the period of image generation rate. The method of generating a composite reconstructed image from a plurality of reconstructed images can include averaging a plurality of pieces of reconstructed image data having the same voxel coordinates. Moreover, the more desirable method can include performing weighted averaging on a plurality of pieces of reconstructed image data having the same voxel coordinates with the quantity of light radiated in each radiation pattern.

Then, the display unit 160 displays composite reconstructed image data.

Here, the sampling period tw1 and the period of image capturing frame rate tw2 are determined as follows.

As mentioned above, due to a restriction imposed by the MPE value, the sampling period tw1 is determined based on the peak power of pulsed light and the radiation area. Then, the number of times of the arithmetic mean operation is determined based on the ratio of the S/N of a photoacoustic signal acquired by radiation of pulsed light performed one time to the S/N of a photoacoustic signal determined by the specified image quality. For example, if the S/N of a photoacoustic signal acquired by radiation of pulsed light performed one time is 1/5 times the S/N of a photoacoustic signal determined by the specified image quality, S/N is required to be five times improved. Therefore, averaging is performed 25 times. For example, if the sampling period tw1 is 0.1 ms, the period of image capturing frame rate is 2.5 ms or more, in other words, the image capturing frame rate is 400 Hz or less.

Moreover, the sampling period tw1 is also restricted by heat generation of semiconductor light-emitting elements. More specifically, if the thermal resistance of the probe is determined, the temperature is determined based on the power consumption of semiconductor light-emitting elements. The sampling period tw1 is made longer in such a manner that the temperature of semiconductor light-emitting elements does not exceed the permissible temperature.

On the other hand, since, if the number of times of the arithmetic mean operation is made large, photoacoustic signals are subjected to the arithmetic mean operation for a long time, in a case where the subject has, for example, a body motion, blurring occurs due to the motion. To reduce motion blur, it is more advantageous to make the number of times of the arithmetic mean operation as small as possible. Specifically, it is desirable to design the number of times of the arithmetic mean operation in such a manner that the motion blur is restricted to 1/2 or less of the specified resolution. For example, assuming that the specified resolution is 0.2 mm and the body motion of the subject is 5 mm/sec, in a case where the sampling period tw1 is 0.1 ms, the number of times of the arithmetic mean operation is set to 200 times or less, in other words, the period of image capturing frame rate tw2 is set to 20 ms or less.

In consideration of such a plurality of conditions, the sampling period tw1 and the period of image capturing frame rate tw2 are determined. Naturally, in a case where it is impossible to satisfy all of the conditions, the priority for the conditions is determined and these parameters are thus determined.

The first exemplary embodiment of the invention has been described with a radiation mode which includes four radiation patterns as illustrated in FIGS. 4A to 4D. The radiation mode is not limited to this example. For example, a radiation mode in which, in each radiation pattern, only one of the semiconductor light-emitting elements 200a to 200h performs light emission can be employed. In this case, the radiation mode includes eight radiation patterns. If such radiation mode is employed, a longer time is required to acquire reconstructed image data about all of the regions. However, it becomes possible to prevent or reduce an influence of photoacoustic waves generated at the subject surface, which may become a cause for noise. Moreover, with regard to a sequence of radiation patterns, radiation patterns according to which the interval of light emission time between adjacent semiconductor light-emitting elements is small are desirable. If the interval of light emission time between adjacent semiconductor light-emitting elements is large, the respective reconstructed images become out of alignment under the influence of a body motion, so that, when combined, the reconstructed images may not be correctly connected.

According to the first exemplary embodiment of the invention, providing a plurality of radiation patterns to cause semiconductor light-emitting elements to perform light emission in a time-division manner enables preventing a decrease in image quality caused by high-intensity photoacoustic waves generated near the subject surface.

Next, a second exemplary embodiment of the invention is described.

As mentioned above, according to the first exemplary embodiment, a good-quality composite reconstructed image can be obtained. However, since a composite reconstructed image is obtained by acquiring a plurality of pieces of reconstructed image data using a plurality of radiation patterns, a long time is required. The second exemplary embodiment of the invention is a configuration capable of updating a composite reconstructed image in a short time even in a radiation mode including a plurality of radiation patterns.

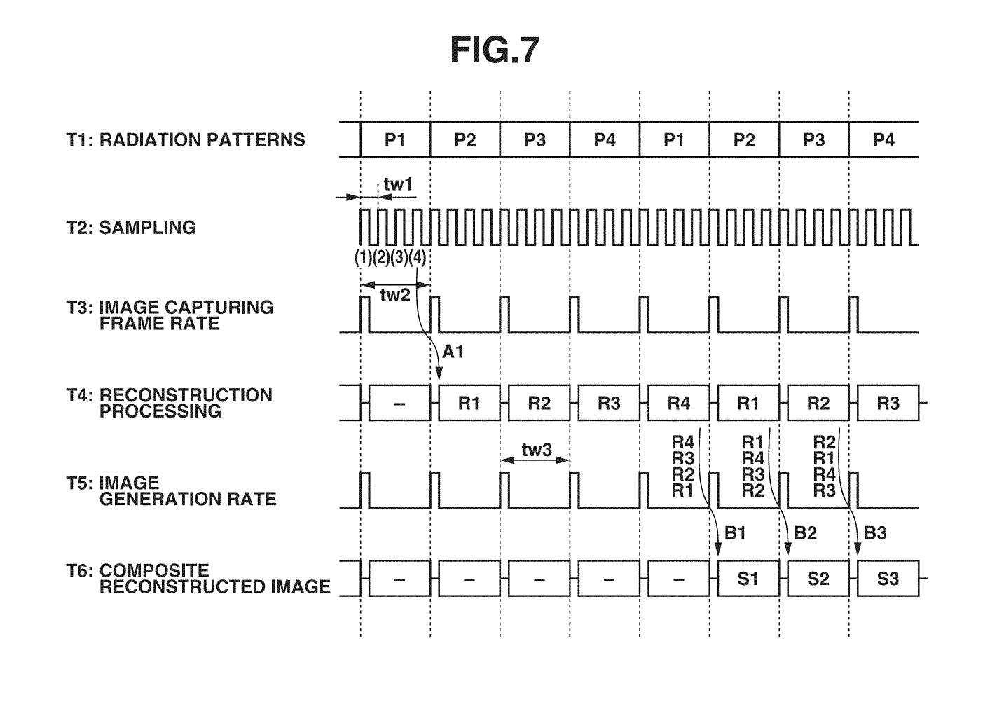

FIG. 7 is a timing chart used to comprehensibly describe an operation in the second exemplary embodiment of the invention. In FIG. 7, the horizontal axis is a time axis. Line T1 to line T6 illustrated in FIG. 7 are similar to those described in the first exemplary embodiment, and are, therefore, omitted from description. In the second exemplary embodiment, as indicated by line T4 illustrated in FIG. 7, the photoacoustic apparatus sequentially calculates pieces of reconstructed image data with the period of image capturing frame rate.

As indicated by line T4 to line T6 illustrated in FIG. 7, the photoacoustic apparatus combines pieces of reconstructed image data R1, R2, R3, and R4 (B1). Then, after calculating a next reconstructed image, the photoacoustic apparatus combines pieces of reconstructed image data R2, R3, R4, and R1 with the period of image capturing frame rate (B2). Then, after calculating a next reconstructed image, the photoacoustic apparatus combines pieces of reconstructed image data R3, R4, R1, and R2 with the period of image capturing frame rate (B3). Repeating the above operation, the photoacoustic apparatus is able to calculate composite reconstructed image data with the period of image capturing frame rate.

In this case, while a composite reconstructed image is formed with pieces of reconstructed image data corresponding to the radiation patterns sequentially updated, there is an advantage of being able to update a portion for which reconstructed image data has been acquired with a minimum delay.

According to the second exemplary embodiment of the invention, as with the first exemplary embodiment, a decrease in image quality caused by a high-intensity photoacoustic signal generated near the subject surface can be prevented. Moreover, updating of a composite reconstructed image can be performed in the shortest amount of time.

A third exemplary embodiment of the invention provides a photoacoustic apparatus having a plurality of radiation modes and capable of switching radiation modes according to an instruction from the user or automatically. The designation of a radiation mode by the user can be performed via the mouse 171 or the keyboard 172 of the input unit 170. Moreover, storing and execution of radiation modes and radiation patterns are performed by the computer 150.

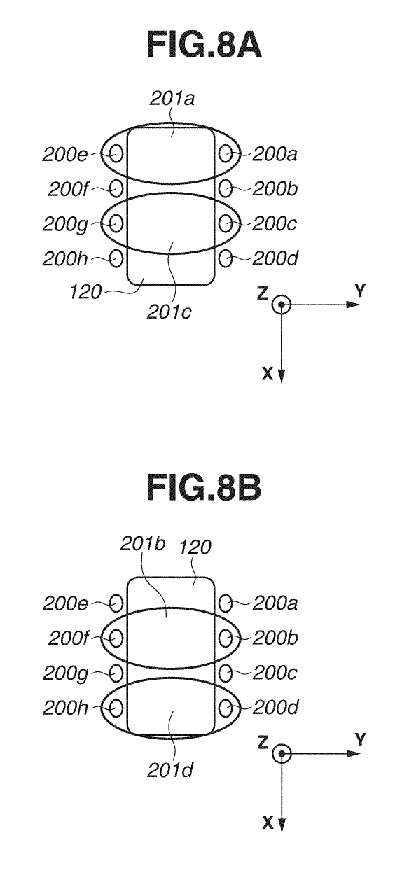

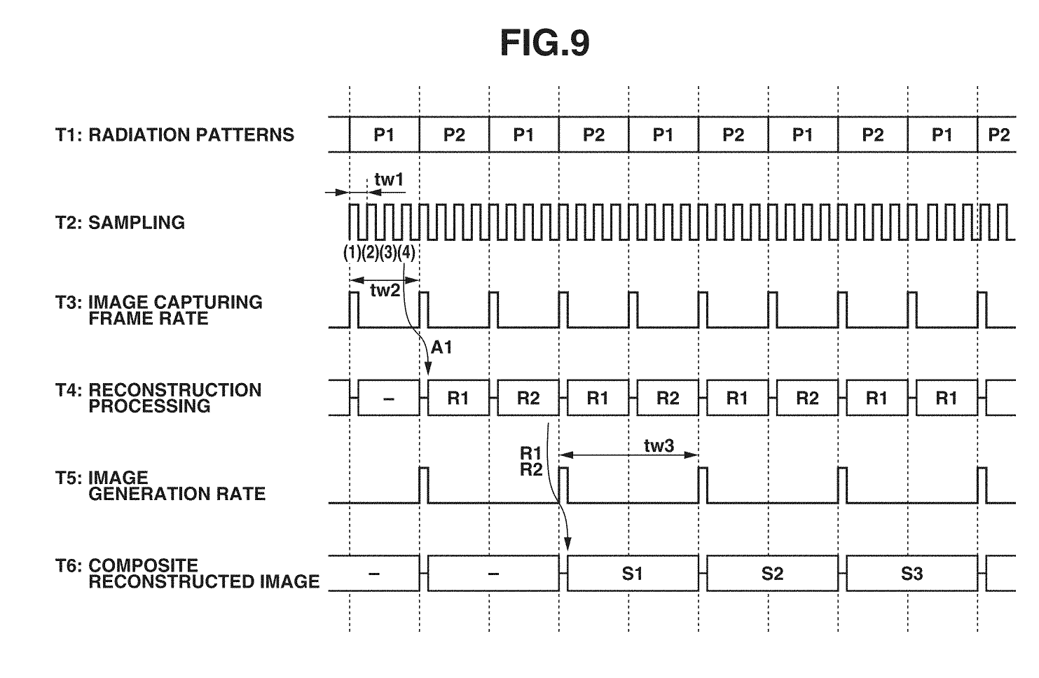

The photoacoustic apparatus according to the third exemplary embodiment has, for example, three radiation modes. Radiation mode 1 serving as the first radiation mode is the radiation mode described in the first exemplary embodiment. In other words, the radiation mode 1 includes four radiation patterns in each of which two of eight semiconductor light-emitting elements perform light emission at the same time. Radiation mode 2 serving as the second radiation mode includes two radiation patterns illustrated in FIGS. 8A and 8B. FIGS. 8A and 8B are diagrams illustrating a plurality of radiation patterns of the radiation mode 2 in the third exemplary embodiment, i.e., a light emission sequence and irradiated regions of the semiconductor light-emitting elements 200a to 200h. The radiation mode 2 is made up of a repetition of two radiation patterns in each of which four semiconductor light-emitting elements perform light emission at the same time. FIG. 9 is a timing chart used to comprehensibly describe an operation in the radiation mode 2. In FIG. 9, the horizontal axis is a time axis. Line T1 to line T6 illustrated in FIG. 9 are similar to those described in the first exemplary embodiment, and are, therefore, omitted from description. In the radiation mode 2, as indicated by line T4 illustrated in FIG. 9, the photoacoustic apparatus sequentially calculates pieces of reconstructed image data with the period of image capturing frame rate. Then, as indicated by line T4 to line T6 illustrated in FIG. 9, the photoacoustic apparatus combines pieces of reconstructed image data R1 and R2, and then calculates composite reconstructed image data with the period of image generation rate.

Radiation mode 3 serving as the third radiation mode is a radiation mode including one radiation pattern in which eight semiconductor light-emitting elements perform light emission at the same time.

Characteristics of such three radiation modes are as follows. The radiation mode 3 is able to irradiate the entire region of the subject with light emission of one radiation pattern. Therefore, the radiation mode 3 is able to obtain a reconstructed image of the entire region of the subject at a speed higher than those of the other radiation modes. Moreover, the radiation mode 3 does not need to perform combining of reconstructed images. In a case where there are few optical absorbers near the subject surface, since deterioration of a reconstructed image is small, the radiation mode 3 is effective. As mentioned above, the radiation mode 1 is a mode capable of reducing deterioration of a reconstructed image in a case where an optical absorber is present near the subject surface. However, since reconstructed images acquired by radiation performed four times are combined to obtain a reconstructed image of the entire region of the subject, the speed of obtaining a reconstructed image becomes low. The radiation mode 2 is a radiation mode intermediate between the radiation mode 1 and the radiation mode 3, and has advantages of both modes.

In a photoacoustic apparatus having such three radiation modes, it is desirable that the photoacoustic apparatus be configured to allow the user to select a radiation mode. For example, in the case of a subject with a low melanin concentration of the pigment contained in the skin, such as the skin of a Caucasian person, the user can select the radiation mode 3. Moreover, in a case where the probe is applied to the surface of the skin of a Negroid person or the skin having a mole, since the melanin concentration of the pigment contained in the skin is high, the user can select the radiation mode 1. Additionally, it is desirable that, with radiation modes being configured to be switchable in real time, the user be allowed to select a radiation mode while viewing the obtained reconstructed image.

Furthermore, it is desirable that a radiation mode be automatically set according to the optical absorption coefficient of the skin. More specifically, the probe 180 can be additionally provided with a camera or reflectance measurement device used to observe the condition of the skin, and a radiation mode can be automatically selected according to the brightness of the skin. Naturally, in a case where the brightness of the skin is low (in a case where the optical absorption coefficient of the skin is large), the radiation mode 1 is selected. Moreover, without use of the camera or reflectance measurement device, the optical absorption coefficient of the skin can be estimated based on the magnitude of a photoacoustic signal on the skin surface received by the ultrasonic wave reception unit 120, so that a radiation mode can be selected based on the estimated optical absorption coefficient. For example, first, a photoacoustic signal is received in the radiation mode 3, and, if a signal at the time corresponding to the skin surface of the acquired photoacoustic signal is large, the computer 150 performs control to select the radiation mode 2, and, if the signal is much larger, the computer 150 performs control to select the radiation mode 1.

As described above, according to the third exemplary embodiment of the invention, an optimal radiation mode can be designated by the user or can be selected automatically according to, for example, the melanin concentration of the skin serving as a subject. As a result, a reconstructed image with less noise can be obtained in the shortest amount of time without depending on the melanin concentration of the skin.

A fourth exemplary embodiment of the invention is another exemplary embodiment of the hand-held type probe.

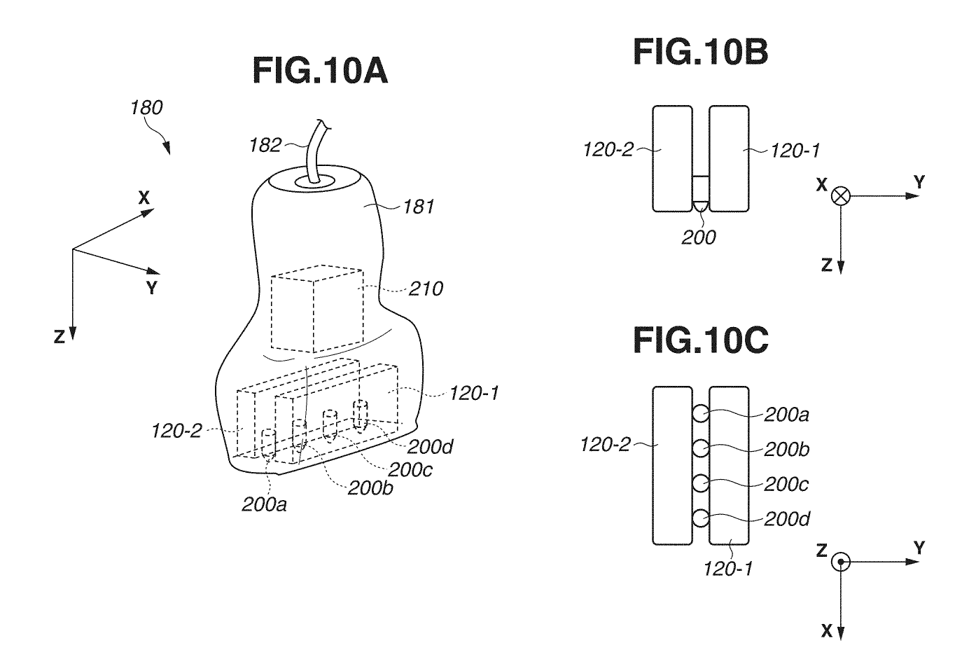

FIGS. 10A, 10B, and 10C are diagrams illustrating a structure of the probe 180 according to the fourth exemplary embodiment. As with FIG. 3A, in FIG. 10A, the probe 180 includes a light radiation unit 200, a driver unit 210, ultrasonic wave reception units 120-1 and 120-2, and a housing 181. The light radiation unit 200 irradiates the subject with pulsed light. The light radiation unit 200 is configured with, for example, a plurality of semiconductor light-emitting elements. As illustrated in FIGS. 10A to 10C, in the fourth exemplary embodiment, each of the ultrasonic wave reception unit 120-1 and the ultrasonic wave reception unit 120-2 is configured with a transducer array. Then, the light radiation unit 200 is mounted while being sandwiched between the ultrasonic wave reception unit 120-1 and the ultrasonic wave reception unit 120-2. The light radiation unit 200 is implemented by semiconductor light-emitting elements 200a to 200d. The semiconductor light-emitting elements 200a to 200d are specifically implemented by four semiconductor lasers. Furthermore, the X-, Y-, and Z-axes in FIGS. 10A to 10C indicate coordinate axes in a case where the probe is left to stand, and are not the axes which limit the orientation of the probe during use thereof.

The probe 180, which is illustrated in FIG. 10A, is connected to the signal acquisition unit 140 via a cable 182. The cable 182 includes a wiring used to supply electric power to the light radiation unit 200, a light emission control signal wiring, and a wiring used to output analog signals output from the ultrasonic wave reception unit 120-1 and the ultrasonic wave reception unit 120-2 to the signal acquisition unit 140.

FIG. 10B is a Y-Z sectional view of a portion including the light radiation unit 200, the ultrasonic wave reception unit 120-1, and the ultrasonic wave reception unit 120-2 of the probe 180. FIG. 10C is a diagram of the portion including the light radiation unit 200, the ultrasonic wave reception unit 120-1, and the ultrasonic wave reception unit 120-2 of the probe 180 as viewed from a contact surface with the subject.

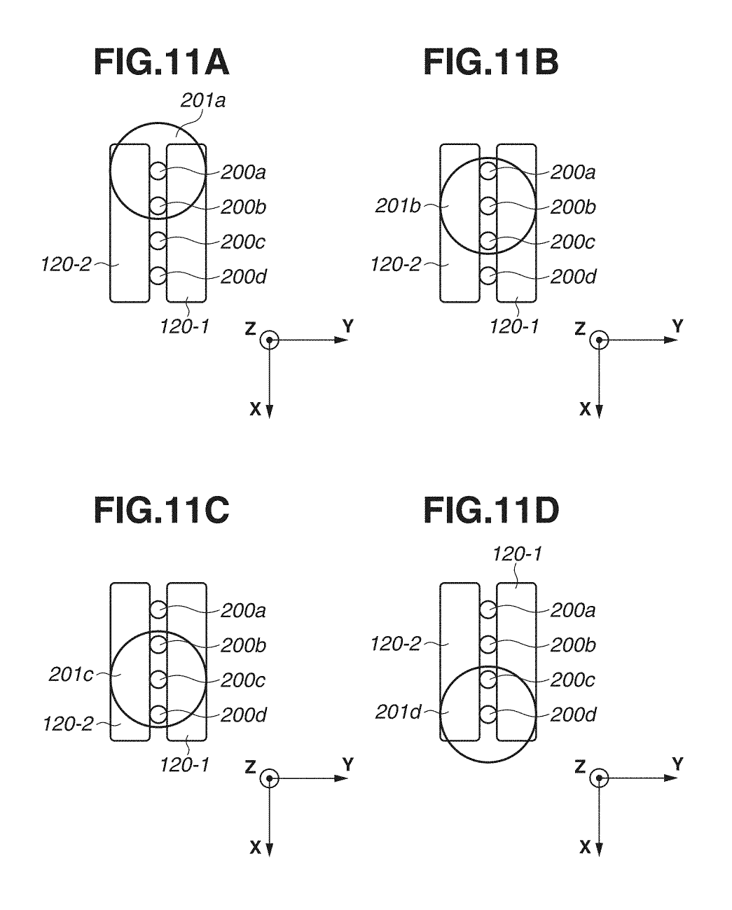

A light emission sequence of the semiconductor light-emitting elements 200a to 200d and irradiated regions thereof are illustrated in FIGS. 11A, 11B, 11C, and 11D. The fourth exemplary embodiment differs from the first exemplary embodiment only in the configuration of the ultrasonic wave reception unit 120-1, and the ultrasonic wave reception unit 120-2 and the configuration of the light radiation unit 200, and performs an operation similar to that of the first exemplary embodiment. Therefore, even in the fourth exemplary embodiment, the light radiation unit 200 irradiates the subject in a radiation mode including four radiation patterns, so that a reconstructed image with less noise can be acquired.

In a case where the light radiation unit (light source) 200 is implemented by a plurality of semiconductor light-emitting elements, any variation of light outputs of the respective semiconductor light-emitting elements induces luminance variation in generation of a composite reconstructed image. In other words, a variation in luminance occurs in a composite reconstructed image. A fifth exemplary embodiment of the invention is a configuration for correcting a variation in luminance of a reconstructed image, which is caused by a variation of light outputs between a plurality of semiconductor light-emitting elements constituting the light radiation unit (light source) 200.

The fifth exemplary embodiment is described with reference to the configuration of the light radiation unit 200 including a plurality of semiconductor light-emitting elements 200a to 200h described in the first exemplary embodiment.

In a case where there is a variation in the quantity of light emission between a plurality of semiconductor light-emitting elements 200a to 200h, corrections are performed based on any of the following correction methods.

One correction method actually measures the quantity of light of an irradiated region for each of a plurality of radiation patterns, and corrects a reconstructed image acquired in each of the plurality of radiation patterns with the reciprocal of the quantity of light of an irradiated region for each of the plurality of radiation patterns. Such a correction can be easily performed in a case where a plurality of semiconductor light-emitting elements performs light emission according to radiation patterns. As illustrated in FIG. 1B, depending on a radiation pattern, a reconstructed image is not affected by light emission of the other semiconductor light-emitting elements. In other words, a reconstructed image acquired in each radiation pattern is not affected by the quantity of light emission of the other semiconductor light-emitting elements, and is, therefore, able to be easily corrected. In this way, the method of actually measuring the quantity of light of an irradiated region for each radiation pattern and correcting a corresponding reconstructed image enables correcting a variation in the quantity of light between semiconductor light-emitting elements in an appropriate manner.

Another correction method is a method of actually acquiring a composite reconstructed image such as a phantom having no variation in optical absorption coefficient and generating correction data in such a manner that an unevenness in luminance does not occur in the composite reconstructed image. Moreover, a further method of actually acquiring a composite reconstructed image with use of a phantom with a known optical absorption coefficient and generating correction data in such a manner that an unevenness in luminance of the composite reconstructed image becomes a luminance corresponding to the known optical absorption coefficient can also be employed. Thus, a method of performing corrections to a composite reconstructed image can be employed.

This correction method is suitable for a case where a portion in which irradiated regions of semiconductor light-emitting elements overlap is large as in the above-mentioned radiation mode 3.

As described in the fifth exemplary embodiment of the invention, even in a case where the light radiation unit (light source) 200 is implemented by a plurality of semiconductor light-emitting elements, a variation in a reconstructed image caused by a variation in light outputs of the plurality of semiconductor light-emitting elements can be corrected in an appropriate manner.

Radiation patterns which are effective in exemplary embodiments of the invention are described. As apparent from FIGS. 1A and 1B, when a semiconductor light-emitting element in a region close to a region to be irradiated for performing reconstruction performs light emission, photoacoustic waves on the subject surface are more likely to be generated at a relatively near place. Since noises that are caused by photoacoustic waves generated at a relatively near place are large, if radiation patterns in which a semiconductor light-emitting element corresponding to a region close to a region to be irradiated for performing reconstruction does not perform light emission are employed, any decrease in image quality can be reduced. In other words, light emission can be performed in such a manner that an adjacent semiconductor light-emitting element is prevented from performing light emission. Specifically, radiation patterns in which semiconductor light-emitting elements located at discrete positions perform light emission at the same time, for example, every other n (n=1, 2, 3, 4, . . . ) semiconductor light-emitting elements perform light emission at the same time, are desirable. On the other hand, as mentioned above, if the interval of light emission time between adjacent semiconductor light-emitting elements is large, the respective reconstructed images become out of alignment under the influence of a body motion, so that, when combined, the reconstructed images may not be correctly connected. Therefore, appropriate radiation patterns are determined based on various conditions according to an actual subject.

The wavelength of light which the light radiation unit 200 emits can include a plurality of wavelengths as mentioned above. If a plurality of wavelengths is employed, oxygen saturation serving as functional information can be calculated. For example, exemplary embodiments of the invention can acquire photoacoustic signals while alternately switching two wavelengths with a period of image generation rate, calculate composite reconstructed image data, and calculate oxygen saturation based on two pieces of composite reconstructed image data. The calculation of oxygen saturation is described in detail in Japanese Patent Application Laid-Open No. 2015-142740, and the detailed description thereof is, therefore, omitted.

Moreover, a photoacoustic apparatus according to an exemplary embodiment of the invention can be additionally provided with the function of transmitting ultrasonic waves from transducers and performing measurement based on reflected waves. In this case, naturally, the light radiation unit 200 does not perform light emission.

In a photoacoustic apparatus according to an exemplary embodiment of the invention, since a plurality of semiconductor light-emitting elements is used as a light source, high-density packaging regarding light radiation positions can be easily implemented. Moreover, since a plurality of electrical signals derived from light radiated onto a subject in positions different from each other and at times different from each other is reconstructed independently from each other to generate a plurality of images and the generated plurality of images is combined, a good-quality image can be obtained.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2017-107062, filed May 30, 2017, which is hereby incorporated by reference herein in its entirety.

What is claimed is:

1. A photoacoustic apparatus comprising:
a plurality of semiconductor light-emitting elements;
an ultrasonic wave reception unit configured to receive ultrasonic waves generated by radiation of light from the plurality of semiconductor light-emitting elements onto a subject and to output an electrical signal based on the received ultrasonic waves;
a control unit configured to control light emission patterns of the plurality of semiconductor light-emitting elements to radiate light onto the subject in positions different from each other and at times different from each other; and
an image generation unit configured to generate a plurality of images, each such image being generated by independently reconstructing a respective electrical signal output by the ultrasonic wave reception unit based on the light radiated onto the subject in a respective one of the positions and at a respective one of the times, and configured to generate a composite image concerning the subject by combining the plurality of images.

2. The photoacoustic apparatus according to claim 1, wherein, when generating the composite image, the image generation unit performs weighting processing based on information concerning a light quantity distribution of an irradiated region of light in the subject, which is determined at least based on the light emission patterns.

3. The photoacoustic apparatus according to claim 1, wherein the image generation unit determines a region in which to reconstruct an image, based on information concerning a light quantity distribution of an irradiated region of light in the subject, which is determined at least based on the light emission patterns.

4. The photoacoustic apparatus according to claim 1, wherein at least one of the light emission patterns is a light emission pattern for causing all of the plurality of semiconductor light-emitting elements to perform light emission.

5. The photoacoustic apparatus according to claim 1, further comprising a mode control unit configured to switch the light emission patterns,
wherein the mode control unit determines the light emission patterns based on information about an instruction from a user or information about an optical absorption coefficient of a surface of the subject.

6. The photoacoustic apparatus according to claim 5, wherein, in a case where the optical absorption coefficient of the surface of the subject is smaller than a predetermined value, the mode control unit selects one of the light emission patterns for causing all of the plurality of semiconductor light-emitting elements to perform light emission.

7. The photoacoustic apparatus according to claim 1, wherein the ultrasonic wave reception unit is configured to include a plurality of ultrasonic transducers arranged in an array-like manner along a first direction.

8. The photoacoustic apparatus according to claim 7, wherein the plurality of semiconductor light-emitting elements is arranged in an array-like manner along the first direction.

9. The photoacoustic apparatus according to claim 1, wherein the photoacoustic apparatus is a hand-held type probe.
